(19) United States
(12) Patent Application Publication
(10) Pub. No.: US 2018/0280373 A1
(43) Pub. Date: Oct. 4, 2018

(54) PALONOSETRON FORMULATIONS AND USES THEREOF

(71) Applicant: LUXENA PHARMACEUTICALS, INC., Santa Clara, CA (US)

(72) Inventors: Xiaodong LI, Sunnyvale, CA (US); George LU, Palo Alto, CA (US); Biao LU, Palo Alto, CA (US)

(21) Appl. No.: 15/880,889

(22) Filed: Jan. 26, 2018

Related U.S. Application Data
(63) Continuation of application No. 15/501,156, filed on Feb. 1, 2017, now abandoned.
(60) Provisional application No. 62/031,872, filed on Aug. 1, 2014.

(51) Int. Cl. A61K 31/473; A61K 9/00

(52) U.S. Cl. CPC A61K 31/473; A61K 9/008; A61K 9/0075

(57) ABSTRACT

The present disclosure provides for palonosetron formulations, such as aerosol formulations of palonosetron for pulmonary delivery. Also provided are uses of the formulation, such as reduction, elimination or prevention of nausea and vomiting associated with chemotherapy, radiation therapy, or surgery. Also provided are novel methods to treat chemotherapy-induced nausea and vomiting (CINV), radiation-induced nausea and vomiting (RINV), and post-operative nausea and vomiting (PONV) using the inhalation formulations.

### CROSS-REFERENCE

This application claims the benefit of U.S. Provisional Application No. 62/031,872, filed on Aug. 1, 2014, which is incorporated herein by reference in its entirety.

### BACKGROUND

Cancer is one of the major causes of death in the modem world. Major therapies to treat cancers include chemotherapy, radiation therapy and surgery. Nausea and vomiting are among the most common side-effects of these treatments. Patients receiving highly emetogenic agents may postpone, or even refuse, potentially curative treatments. Palonosetron is a 5-HT₃ receptor antagonist used mainly as an antiemetic in the prevention and treatment of nauseas and vomiting induced by chemotherapy, radiation therapy and surgery. Palonosetron is believed to block 5-HT₃ receptors in the chemoreceptor trigger zone. FIG. 1 shows the exemplary skeletal formula of palonosetron.

Currently, palonosetron is administered either through injection (slow IV or IM) or as oral tablets. Injection of palonosetron, although effective in reducing or preventing nausea and vomiting, is inconvenient, invasive and causes pain to the patients. Existing forms of oral palonosetron tablets can be difficult to swallow and may be undesirable to some patients requiring anti-emetic therapy, especially those patients who have severe nausea or vomiting.

Thus, there remains a need for new formulations and for novel methods to administer palonosetron. The formulations, and methods described herein are directed toward this end.

### BRIEF SUMMARY

In one aspect, the present disclosure provides for a formulation comprising palonosetron or a pharmaceutically acceptable salt thereof, wherein the formulation exhibits a minimum fine particle fraction (FPF) of at least 40%. In some embodiments, the palonosetron or a pharmaceutically acceptable salt thereof is present in an amount of about 0.01% to about 12% of a total weight of the formulation. In some embodiments, the palonosetron or a pharmaceutically acceptable salt thereof is present in an amount of about 0.1% to about 5% of the total weight of the formulation, for example about 0.6%, about 2%, or about 4% of the total weight of the formulation. In some embodiments, the formulation comprises an excipient that comprises a fine powder and a coarse powder. In some embodiments, the fine powder and coarse powder are of the same substance. In some embodiments, the fine powder and coarse powder are lactose. In some embodiments, the fine powder has a mass median diameter of less than 0.1-50 microns. In some embodiments, the coarse powder has a mass median diameter of about 50-500 microns. In some embodiments, the fine powder and coarse powder are present in a mass ratio of 1:1 to 1:50. In some embodiments, a mass median aerodynamic diameter (MMAD) of the palonosetron or a pharmaceutically acceptable salt thereof is less than 5 microns. In some embodiments, the MMAD of the palonosetron or a pharmaceutically acceptable salt thereof is less than 3 microns. In some embodiments, the average particle size of the palonosetron or a pharmaceutically acceptable salt thereof is less than 10 microns. In some embodiments, the average particle size of the palonosetron or a pharmaceutically acceptable salt thereof is less than 5 microns. In some embodiments, the pharmaceutically acceptable salt thereof is palonosetron hydrochloride. In some embodiments, the formulation is suitable for inhalation. In some embodiments, the formulation is suitable for pulmonary delivery. In some embodiments, the formulation is for nasal administration. In some embodiments, the formulation is for buccal administration. In some embodiments, the formulation is in a form of aerosol. In some embodiments, the formulation is in a form of powder. In some embodiments, the average particle size of the formulation is about 200 microns or less. In some embodiments, the average particle size of the formulation is about 50 microns or less. In some embodiments, the formulation comprises a propellant. In some embodiments, the formulation comprises a propellant that is 1,1,1,2-tetrafluoroethane (P134a), 1,1,1,2,3,3,3-heptafluoro-n propane (P227), 2H,3H-perfluoropentane (HPFP), or any combination thereof. In some embodiments, the formulation comprises an excipient that comprises galactose, mannose, sorbose, lactose, glucose, trehalose, raffinose, maltodextrins, dextrans, mannitol, xylitol, or any combination thereof. In some embodiments, the formulation comprises an excipient that comprises alanine, glycine, tryptophan, tyrosine, leucine, phenylalanine, or any combination thereof. In some embodiments, the formulation comprises an excipient that comprises sorbitan trioleate, isopropyl myristate, lecithin, oleic acid or oleic acid esters, propylene glycol, isopropyl laurate, polyvinylpyrrolidone (PVP), dipalmitoylphosphatidylcholine (DPPC), 2,6-di-tert-butyl-p-cresol (DBPC), or any combination thereof. In some embodiments, the formulation comprises a solvent that is C2-6 alcohols, polyols, cineole, citral, lactic acid oligomers, poly(ethylene glycols), or any combination thereof. In some embodiments, the formulation is contained in a capsule, a blister, or a canister. In some embodiments, upon pulmonary delivery to a subject, the formulation exhibits an AUC of palonosetron about the same as that obtained following intravenous delivery of palonosetron. In some embodiments, upon pulmonary delivery to a subject, the formulation exhibits an AUC of palonosetron equal or higher of that obtained following oral delivery of palonosetron. In some embodiments, upon pulmonary delivery to a subject, the formulation exhibits an AUC of palonosetron of about 1.5 times or more of that obtained following intravenous or oral delivery of palonosetron. In some embodiments, upon pulmonary delivery to a subject, the formulation exhibits a Cmax of palonosetron equal or less of that obtained following intravenous delivery of palonosetron. In some embodiments, upon pulmonary delivery to a subject, the formulation exhibits a Cmax of palonosetron equal or higher of that obtained following oral delivery of palonosetron. In some embodiments, upon pulmonary delivery to a subject, the formulation exhibits a Cmax of palonosetron of about 1.5 times or more of that obtained following oral delivery of palonosetron. In some embodiments, upon pulmonary delivery to a subject, the formulation exhibits a Tmax of palonosetron about the same as that obtained following intravenous delivery of palonosetron. In some embodiments, upon pulmonary delivery to a subject, the formulation exhibits a Tmax of palonosetron equal or less of that obtained following oral delivery of palonosetron. In some embodiments, upon pulmonary delivery to a subject, the formulation exhibits a Tmax of palonosetron of about 0.5 times or less of that obtained following oral delivery of palonosetron.

The present disclosure also provides for a method of reducing or preventing nausea or vomiting in a subject, comprising administering a formulation herein to the subject. In some embodiments, the nausea or vomiting is chemotherapy-induced or radiation-induced. In some embodiments, the nausea or vomiting is post-operative nausea or vomiting. In some embodiments, the subject is mammal, for example human. In some embodiments, the subject is a cancer patient. In some embodiments, the subject is a patient who receives an operation.

The present disclosure also provides for a method of making the formulation herein. In some embodiments, the method comprises spray drying. In some embodiments, the method comprises a mechanical micronization process. In some embodiments, the method comprises a supercritical fluid process. In some embodiments, the method comprises direct controlled crystallization. In some embodiments, the method comprises blending all of the palonosetron or a pharmaceutically acceptable salt thereof, a fine powder of an excipient, and a coarse powder of the excipient together at the same time. In some embodiments, the method comprises first blending the palonosetron or a pharmaceutically acceptable salt thereof with a fine powder of an excipient, a resulting mixture of which is further blended with a coarse powder of the excipient. In some embodiments, the method comprises first blending the palonosetron or a pharmaceutically acceptable salt thereof with a coarse powder of an excipient, a resulting mixture of which is further blended with a fine powder of the excipient. In some embodiments, the method comprises blending the palonosetron or a pharmaceutically acceptable salt thereof with a fine powder of the excipient and a coarse powder of the excipient separately, two resulting mixtures of which are further blended. In some embodiments, the method comprises first blending a fine powder of an excipient and a coarse powder of the excipient, a resulting mixture of which is further blended with palonosetron or a pharmaceutically acceptable salt thereof.

In some embodiments, provided herein are novel aerosol inhalation formulations of palonosetron for pulmonary delivery; and uses thereof in the reduction, elimination or prevention of nausea and vomiting associated with chemotherapy, radiation therapy and surgery. Also provided are methods to treat chemotherapy-induced nausea and vomiting (CINV), radiation-induced nausea and vomiting (RINV), and post-operative nausea and vomiting (PONV) using the inhalation formulations.

In one aspect, the present disclosure provides novel aerosol formulations comprising palonosetron useful for pulmonary delivery to a subject. In some embodiments, the aerosol formulations are administered by inhalation. In some embodiments, the aerosol formulations are delivered into the circulation via the pulmonary tract. In some embodiments, the subject is a patient such as a cancer patient.

In one aspect, the present disclosure provides pharmaceutical aerosol inhalation formulations comprising palonosetron.

In one aspect, the aerosol formulations described herein are useful for the reduction, elimination or prevention of various medical conditions including chemotherapy-induced nausea and vomiting (CINV), radiation-induced nausea and vomiting (RINV), and post-operative nausea and vomiting (PONV).

In one aspect, the present disclosure provides methods of treating a condition of nausea or vomiting, wherein the method comprises pulmonary administration of a pharmaceutically acceptable amount of the aerosol formulations described herein, and wherein the aerosol formulations are administered into the pulmonary tract by inhalation.

In one aspect, the present disclosure provides methods for pulmonary delivery of palonosetron to a subject that comprise having the subject inhale a pharmaceutically acceptable amount of the aerosol formulation described herein through the subject's mouth into the circulation via the pulmonary tract. In some embodiments, the subject is a cancer patient.

In one aspect, the present disclosure provides a method for pulmonary delivery of palonosetron to a subject, where the method comprises having the subject inhale a pharmaceutically acceptable amount of the aerosol formulation described herein through the subject's nose into the circulation via the pulmonary tract. In some embodiments, the subject is a cancer patient.

In one aspect, with respect to the aerosol formulations or methods described herein, the pulmonary administration of the aerosol formulations minimizes the first pass metabolism before the drug reaches the target receptors since there is rapid transport from the alveolar epithelium into the circulation. In addition, the pulmonary administration of the aerosol formulations described herein by inhalation avoids gastrointestinal intolerance for nausea and vomiting sufferers.

### BRIEF DESCRIPTION OF THE DRAWINGS

FIG. 1: Exemplary Skeletal Formula of Palonosetron

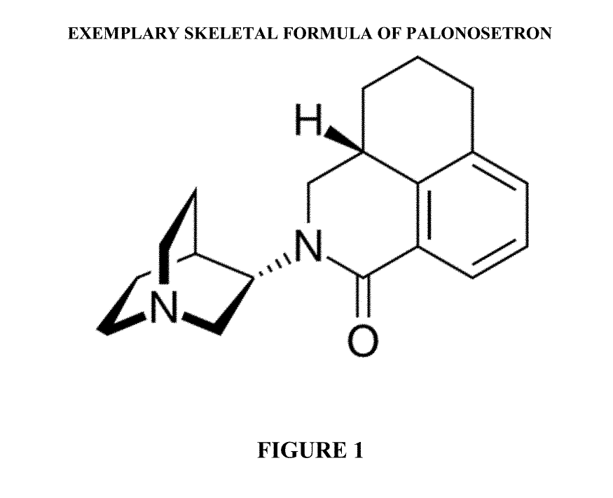

FIG. 2: Particle Size Distribution of Jet Milled Palonosetron Powder

FIG. 3: The Aerodynamic Particle Size Distribution (APSD) of the Exemplary Formulation I

FIG. 4. The APSD of the Exemplary Formulation II

FIG. 5. The APSD of the Exemplary Formulation III

### DETAILED DESCRIPTION

The present disclosure provides palonosetron formulations, for example aerosol formulations comprising palonosetron. In some embodiments, the formulation is for pharmaceutical use. In some embodiments, the formulation is suitable for inhalation. In some embodiments, the formulation is suitable for pulmonary delivery. In some embodiments, the palonosetron is in a form of powder. In some embodiments, the formulations are delivered into the circulation via pulmonary tract. A subject to whom the formulations are administered may be a mammal, such as a human. In some embodiments, the subject is a patient, for example a cancer patient.

The term “about” means a numeric indication that is plus or minus 15% of the referenced number.

The term “a” or “an” means one or more, unless indicated otherwise.

In some embodiments, the palonosetron is present in an amount of about 0.01% to about 25% of a total weight of a formulation described herein, for example about: 0.01-20%, 0.01-15%, 0.01-10%, 0.01-8%, 0.01-6%, 0.01-4%, 0.01-2%, 0.1-25%, 0.1-20%, 0.1-15%, 0.1-10%, 0.1-8%, 0.1-6%, 0.1-4%, or 0.1-2%. In some embodiments, the palonosetron is present in an amount of about: 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of a total weight of a formulation described herein,

In some embodiments, a palonosetron formulation described herein exhibits an fine particle fraction (FPF) of: at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, for example at least 40%. In some embodiments, a palonosetron formulation described herein exhibits a fine particle fraction (FPF) of about: 10%, 15%, 20%, 25%, 30%, 35%, 40%, 45%, 50%, 55%, 60%, 65%, 70%, 75%, 80%, 85%, or 90%, for example about 50%.

In some embodiments, a pharmaceutical excipient in a formulation described herein comprises one, two, or more of: a mono- or disaccharide, such as glucose, lactose, lactose monohydrate, sucrose, trehalose; a sugar alcohol, such as mannitol or xylitol; polylactic acid; starch; or cyclodextrin. Other suitable excipients include inorganic salts such as sodium chloride, sodium bicarbonate, calcium carbonate, organic salts such as sodium lactate, organic compounds such as polysaccharides, liposomes, polymers, conjugate excipients, or any combination thereof.

In one aspect, a pharmaceutical excipient comprises a coarse powder and a fine powder. In some embodiments, the coarse powder and fine powder are of the same substance, such as lactose. In some embodiments, the coarse powder and fine powder are of difference substance, such as lactose and glucose. In some embodiments, the fine powder has a mass median diameter of less than 20 microns, for example: less than 15 microns, less than 10 microns, less than 5 microns, or less than 1 micron. In some embodiments, the coarse powder has a mass median diameter of about 20-100 microns, for example about: 20-90 microns, 25-85 microns, 30-80 microns, 35-75 microns, 40-70 microns, 45-65 microns, 50-60 microns, or 50-55 microns. In some embodiments, the coarse powder has a mass median diameter of about: 50, 55, or 60 microns. In some embodiments, the fine powder and coarse powder are present in a mass ratio of 1:1 to 1:50, for example about: 1:11, 1:12, 1:13, 1:14, 1:15, 1:16, 1:17, 1:18, 1:19, 1:20, 1:30, 1:40, or 1:50. In some embodiments, the fine powder and coarse powder are present in a mass ratio of 1:1 to 1:10, for example about: 1:1, 1:2, 1:3, 1:4, 1:5, 1:6, 1:7, 1:8, 1:9, or 1:10.

In some embodiments, an average particle size of palonosetron in a formulation described herein is from about 0.05 to about 20 microns, for example about: 0.1-20 microns, 0.1-15 microns, 0.1-10 microns, 0.1-5 microns, 1-20 microns, 1-15 microns, 1-10 microns, 1-5 microns, 1-3 microns, or 2-3 microns. In some embodiments, an average particle size of palonosetron in a formulation described herein is <20 microns, <15 microns, <10 microns, <5 microns, <4 microns, <3 microns, <2 microns, or <1 microns.

In some embodiments, with respect to the aerosol inhalation formulation, the mass median aerodynamic diameter (MMAD) of powdered palonosetron is from about 0.05 to about 20 microns, for example about: 0.1-20 microns, 0.1-15 microns, 0.1-10 microns, 0.1-5 microns, 1-20 microns, 1-15 microns, 1-10 microns, 1-5 microns, 1-3 microns, or 2-3 microns. In some embodiments, the mass median aerodynamic diameter (MMAD) of powdered palonosetron in the aerosol formulation described herein is <20 microns, <15 microns, <10 microns, <5 microns, <4 microns, <3 microns, <2 microns, or <1 microns.

In some embodiments, a formulation herein comprises one or more of amino acids, peptides, or derivatives thereof, which are physiologically acceptable and give acceptable release of the active particles on inhalation. Suitable amino acids may include leucine, isoleucine, lysine, valine, methionine, and phenylalanine, all of which can be in L- or D-form. Suitable salt or a derivative of an amino acid may include aspartame or acesulfame K.

In some embodiments, a formulation herein comprises one or more phospholipids, for example Lecithin, DPPC (dipalmitoyl phosphatidylcholine), PG (phosphatidylglycerol), dipalmitoyl phosphatidylethanolamine (DPPE), dipalmitoyl phosphatidylinositol (DPPI), 1-palmitoyl-2-oleoyl-SN-glycero-3-phosphoglycerol (POPG), phosphoglycerides such as disteroylphosphatidylcholine, diarachidoylphosphatidylcholine dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, long-chain saturated phosphatidylinositols, or any combination thereof. The phospholipids may have acyl substituents on the phosphatidyl groups. As in their natural counterparts, the acyl groups may comprise identical or different, saturated or unsaturated acyl radicals, generally C14-22, especially C16-20, acyl radicals. The phospholipids may comprise, by way of acyl radicals, the saturated radicals palmitoyl C16:0 and stearoyl C18:0 and/or the unsaturated radicals oleoyls C18:1 and C18:2. In some embodiments, the phospholipid has diacyl substitution. In some embodiments, the phospholipids herein comprise two identical saturated acyl radicals, especially dipalmitoyl and distearoyl or a mixture of phospholipids in which such radicals predominate, in mixtures in which dipalmitoyl is the major diacy component. Thus, phosphatidyl choline (PC) and PG may be used may be used with the same diacylphosphatidyl profile as in PC and PG extracted from human or animal or vegetable sources, but if synthetic sources are used the dipalmitoyl component may predominate, as in the DPPC mentioned above.

In some embodiments, a formulation herein comprises a metal stearate, or a derivative thereof, for example, sodium stearyl fumarate or sodium stearyl lactylate, zinc stearate, magnesium stearate, calcium stearate, sodium stearate or lithium stearate.

In some embodiments, a formulation herein comprises one or more surface active materials, for example materials that are surface active in the solid state, which may be water soluble or water dispersible, for example lecithin or soya lecithin, or substantially water insoluble, for example solid state fatty acids such as oleic acid, lauric acid, palmitic acid, stearic acid, erucic acid, behenic acid, or derivatives (such as esters and salts) thereof such as glyceryl behenate. Specific examples of such materials are: phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the additive may be cholesterol.

In some embodiments, a formulation herein comprises sodium benzoate, hydrogenated oils (e.g., those solid at room temperature), talc, titanium dioxide, aluminium dioxide, silicon dioxide, microcrystalline cellulose, or tribasic calcium phosphate (TCP).

In one aspect, the aerosol formulations described herein are useful for the reduction, elimination, or prevention of nausea and vomiting associated with various medical conditions including chemotherapy-induced nausea and vomiting (CINV), radiation-induced nausea and vomiting (RINV), and post-operative nausea and vomiting (PONV).

In one aspect, the aerosol formulations described herein are administered by subjects via an inhaler allowing palonosetron to enter the circulation rapidly.

In one aspect, the aerosol formulations described herein provide a novel route of administration of palonosetron to subjects who have nausea or vomiting and not willing to or not able to swallow or to be injected.

In one aspect, the aerosol formulations described herein contains palonosetron that is in a solute form. In one aspect, the aerosol formulations described herein contains palonosetron that is in a powdered form.

In one aspect, the aerosol formulations described herein contains palonosetron that is in a powdered form, and the powdered palonosetron is in a dry powder form.

In one aspect, the aerosol formulations described herein contains palonosetron that is in a powdered form, and the powdered palonosetron is in a suspension. In one aspect, the powdered palonosetron suspension is in a liquid selected from a group consisting of propellants, hybrid propellants, propellants with stabilizers, propellants with surfactants, propellants with diluents, propellants with cosolvents, water, buffer, and combinations thereof.

In one aspect, the aerosol formulations described herein contains palonosetron that is a solute in a solution, and the solvent is selected from a group consisting of propellants, hybrid propellants, cosolvents, cosolvent mixture, organic solvents, water, buffers, and combinations thereof.

In some embodiments, when the palonosetron in the aerosol formulations is in a powdered form, the powdered palonosetron is produced by one or more particle engineering processes. For example, the powdered palonosetron may be produced by a mechanical micronization operation selected from the group consisting of crushing, cutting, bashing, milling, and grinding. In some embodiments, the powdered palonosetron is produced by a precipitation process, such as spray drying, solution precipitation, lyophilization, or combinations of the foregoing. In some embodiments, the powdered palonosetron is produced by one of more precipitation processes followed by one or more mechanical micronization processes.

In some embodiments, the powdered palonosetron of the aerosol formulations is produced by a spray drying process. The spray drying process may be followed by a cyclone separation filtering process.

In some embodiments, the powdered palonosetron of the aerosol formulations is produced by a direct controlled crystallization process. The direct controlled crystallization process may utilize an antisolvent precipitation technique. Moreover, the size range of the crystallines may be controlled by one or more growth-retarding stabilizing additives.

In some embodiments, the powdered palonosetron of the aerosol formulations is produced by a supercritical fluid process. The supercritical fluid process is selected from the group consisting of rapid expansion of supercritical solution (RESS), solution enhanced diffusion (SEDS), gas-anti solvent (GAS), supercritical antisolvent (SAS), precipitation from gas-saturated solution (PGAS), precipitation with compressed antisolvent (PCA) and aerosol solvent extraction system (ASES).

In some embodiments, with respect to the aerosol formulations, the powdered palonosetron is produced by supercritical fluid process, and the process is rapid expansion of supercritical solution (RESS) process. In some embodiments, the process is solution enhanced diffusion (SEDS) process. In some embodiments, the process is gas-anti-solvent (GAS) process. In some embodiments, the process is supercritical-anti-solvent (SAS) process. In some embodiments, the process is precipitation from gas-anti-solvent (PGAS) process. In some embodiments, the process is precipitation with compressed anti-solvent (PCA) process. In some embodiments, the process is aerosol solvent extraction system (ASES) process. In some embodiments, the process is any combinations of the foregoing.

In some embodiments, with respect to the aerosol formulations, the powdered palonosetron is produced by a supercritical fluid process, and the supercritical fluid process is rapid expansion of supercritical solution process.

In some embodiments, with respect to the aerosol formulations, the mean geometric diameter of powdered palonosetron is at least 0.01 microns, at least 0.05 microns, at least 0.1 microns, at least 0.25 microns, at least 0.5 microns, at least 0.75 microns, at least 0.9 microns, at least 1 microns, at least 1.25 microns, at least 1.5 microns, at least 1.75 microns, or even at least 2.0 microns. The mean geometric diameter of powdered palonosetron is at most 20 microns, at most 15 microns, at most 12 microns, at most 10 microns, at most 9 microns, at most 8 microns, at most 7.5 microns, at most 7 microns, at most 6.5 microns, at most 6.0 microns, at most 5.75 microns, at most 5.5 microns, at most 5.25 microns, at most 5.0 microns, at most 4.75 microns, at most 4.5 microns, at most 4.25 microns, at most 4.0 microns, at most 3.75 microns, at most 3.5 microns, at most 3.25 microns, and even at most 3.0 microns. The mean geometric diameter of powdered palonosetron generally ranges from between 0.05 and 30 microns, for example between 0.1 and 20 microns, between 0.2 and 15 microns, between 0.3 and 10 microns, and for example between 0.5 and 5 microns. Advantageously, the mean geometric diameter of powdered palonosetron is between 1 and 3 microns.

In some embodiments, with respect to the aerosol formulations, the mean geometric diameter of powdered palonosetron is between 0.05 and 20 microns, for example between 0.5 and 4 microns, for example between 1 and 3 microns.

In some embodiments, with respect to the aerosol formulations, the powdered palonosetron has an MMAD of at least 0.01 microns, at least 0.05 microns, at least 0.1 microns, at least 0.25 microns, at least 0.5 microns, at least 0.75 microns, at least 0.9 microns, at least 1 microns, at least 1.25 microns, at least 1.5 microns, at least 1.75 microns, or even at least 2.0 microns. The MMAD of powdered palonosetron is at most 30 microns, at most 20 microns, at most 15 microns, at most 10 microns, at most 9 microns, at most 8 microns, at most 7.5 microns, at most 7 microns, at most 6.5 microns, at most 6.0 microns, at most 5.75 microns, at most 5.5 microns, at most 5.25 microns, at most 5.0 microns, at most 4.75 microns, at most 4.5 microns, at most 4.25 microns, at most 4.0 microns, at most 3.75 microns, at most 3.5 microns, at most 3.25 microns, and even at most 3.0 microns. Generally, the MMAD of the powdered palonosetron is between 0.05 and 30 microns, for example between 0.1 and 20 microns, between 0.2 and 15 microns, for example between 0.3 and 10 microns, between 0.5 and 5 microns, and especially between 1 and 3 microns. In some embodiments, with respect to the aerosol formulations, the powdered palonosetron has an MMAD between 0.05 and 20 microns, for example between 0.5 and 4 microns, and for example between 1 and 3 microns.

In some embodiments, with respect to the aerosol formulations, the mean geometric diameter and the MMAD of powdered palonosetron are similar. In other embodiments, the mean geometric diameter and the MMAD of powdered palonosetron are different. In some embodiments, where the mean geometric diameter and the MMAD of powdered palonosetron are different, the difference is due to the morphology of the palonosetron particles.

In some embodiments, the powdered palonosetron may be a solvate, hydrate, organic salt, inorganic salt, ester, or free base. The powdered palonosetron may also be amorphous, crystalline, or polymorphous. For example, the palonosetron is a chloride, bromide, iodide, mesylate, methanesulphonate, para-toluenesulphonate, or methyl sulphate salt. For example, the palonosetron is in the form of a hydrochloride, anhydrous, monohydrate or dihydrate.

In some embodiments, the palonosetron particles of the aerosol formulations are amorphous.

In some embodiments, the palonosetron particles of the aerosol formulations are crystallines. In some embodiments, the shape of the palonosetron particles is one of the group consisting of spherical, ellipsoidal, cubical, diamond, rectangular, orthorhombic, triangular, hexagonal, needlelike, and porous. For example, the palonosetron particles of the aerosol formulations are spherical.

In some embodiments, the palonosetron particles of the aerosol formulations are polymorphous. In some embodiments, the shapes of the palonosetron particles are two of more from the group consisting of spherical, ellipsoidal, cubical, diamond, rectangular, orthorhombic, triangular, hexagonal, needlelike, and porous.

In some embodiments, with respect to the aerosol formulations, the proportion of palonosetron particles with aerodynamic diameters less than 5 μm is at least 5%, at least 10%, at least 15%, at least 20%, at least 25%, at least 30%, at least 35%, at least 40%, at least 45%, at least 50%, at least 55%, at least 60%, at least 70%, and for example at least 70%. In some embodiments, the proportion of palonosetron particles with aerodynamic diameters less than 5 μm is at most 100%, at most 99%, at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 17.5%, at most 15%, and even at most 12.5%.

In some embodiments, with respect to the aerosol formulations, the proportion of palonosetron particles with aerodynamic diameters less than 5 μm is 10% to 100%, for example from 70% to 100%. In some embodiments, the proportion of palonosetron particles with aerodynamic diameters less than 5 μm is from 20 to 80%, for example from 30% to 70%. In a further embodiment, the proportion of palonosetron particles with aerodynamic diameters less than 5 μm is 10% to 30%.

In some embodiments, the palonosetron in the aerosol formulations described herein exhibits a respirable fraction of 10% or more, for example 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, or 90% or more.

In some embodiments, the aerosol formulations do not comprise an excipient.

In some embodiments, the aerosol formulations comprise a pharmaceutically acceptable excipient. The excipient is any excipient acceptable for pulmonary delivery. For example, the excipient is any inhalable excipient.

In aerosol formulations containing an excipient, the excipient is selected from the group consisting of carbohydrates, amino acids, polypeptides, buffers, salts, polyalcohols, lipids, antioxidants, and mixtures thereof. In some embodiments, the excipient is selected from the group consisting of galactose, mannose, sorbose, lactose, glucose, trehalose, raffinose, maltodextrins, dextrans, mannitol, xylitol, and mixtures thereof. In some embodiments, the excipient is selected from the group consisting of alanine, glycine, tryptophan, tyrosine, leucine, phenylalanine, and mixtures thereof. In some embodiments, the excipient is selected from the group consisting of oleates, stearates, myristates, alkylethers, alkyl arylethers, sorbates, polyvinylpyrrolidone (PVP), 2,6-di-tert-butyl-p-cresol (DBPC), and mixtures thereof. In some embodiments, the excipient is selected from the group consisting of 1,1,1,2-tetrafluoroethane (P134a), 1,1,1,2,3,3,3-heptafluoro-n propane (P227), 2H, 3H-perfluoropentane (HPFP) and mixtures thereof. In some embodiments, the excipient is any combinations of the foregoing.

In some embodiments, the aerosol formulations are pressurized metered dose formulations. In some embodiments, the aerosol formulations are dry powder formulations. In some embodiments, the aerosol formulations are nebulizer formulations.

### Dry Power Formulations

In one aspect, the formulation is a dry powder formulation containing an excipient, where the excipient is lactose, glucose, or a mixture of lactose and glucose.

In some embodiments, the dry powder formulations containing a pharmaceutically acceptable excipient, the excipient consists of powders with an average particles size of <5 to 500 microns, from 1 to 150 microns, or from 5 to 100 microns. The excipient may consists of powders of the same substance with an average particle size of <5 to 500 microns, from 1 to 150 microns, or from 5 to 100 microns. There may also be a mixture of powders in which the average particle size is from <5 to 500 microns, from 1 to 150 microns, or from 5 to 100 microns.

In some embodiments, where the dry powder formulations further comprise a pharmaceutically acceptable excipient and the excipient consists of powders with an average particle size of <5 to 500 microns, the excipient may be a mixture of the same substance with different particle size distributions. For example, in one embodiment the pharmaceutically acceptable excipient having an average particle size of <5 to 500 microns with different particle size distributions is a mixture of coarser powders and finer powders of the same substance, where the finer powders have an average particle size from <5 to 50 microns and the coarser powders have an average particle size of 50 to 500 microns. The finer powders may have an average particle size from <5 to 45 microns, from 10 to 40 microns, from 15 to 35 microns, or from 20 to 30 microns, while the coarser powders may have an average particle size from 60 to 90 microns, from 65 to 85 microns, or from 70 to 80 microns. Alternatively, the finer powders may have an average particle size from 1 to 10 microns, from 1 to 7.5 microns, from 1 to 5 microns, or from 2 to 5 microns, while the coarser powders may have an average particle size from 20 to 60 microns, from 20 to 25 microns, from 30 to 60 microns, from 40 to 60 microns, or from 50 to 60 microns. In some embodiments, the coarser powders have an average particle size from 50 to 90 microns, from 65 to 85 microns, or from 70 to 80 microns. The proportion of finer excipient powders may be 0.1% to 99% of the total amount of excipient powders.

In some embodiments, with respect to the dry powder formulations, the pharmaceutically acceptable excipient having an average particle size of <5 to 500 microns with different particle size distributions is a mixture of finer powders, coarser powders, and much coarser powders of the same substance, where the finer powders have an average particle size of <5 to 20 microns, the coarser powders have an average particles size of 20 to 60 microns, and the much coarser powders have an average particles size of 60 to 500 microns. For example, the finer powders have an average particle size of <5 to 10 microns, the coarser powders have an average particles size of 25 to 45 microns, and the much coarser powders have an average particles size of 75 to 90 microns. The proportion of finer excipient powders may be 0.1% to 99% of the total amount of excipient powders.

In some embodiments, in the dry powder formulations, the pharmaceutically acceptable excipient may be a mixture of different substances with similar particle size distributions in which the average particle size is from <5 to 500 microns or from 5 to 100 microns.

Advantageously, the pharmaceutically acceptable excipient in the dry powder formulations is a mixture of different substances with different particle size distributions in which the average particle sizes are from <5 to 500 microns. Namely, the pharmaceutically acceptable excipient of the dry powder formulations is a mixture of finer powders having an average particle size of <5 to 50 microns and coarser powders with an average particles size of 50 to 500 microns; the finer powders and the coarser powders being different substances. The proportion of finer excipient powders may be 0.1% to 99%, for example about: 1-90%, 1-80%, 1-70%, 1-60%, 1-50%, 1-40%, 1-30%, 1-20%, 1-10%, 10-20%, 10-30%, 10-40%, 10-50%, 10-60%, 10-70%, 10-80%, or 10-90%, of the total amount of excipient powders.

In some embodiments, the pharmaceutically acceptable excipient of the dry powder formulations is a mixture of finer powders having an average particle size of <5 to 20 microns, coarser powders having an average particles size of 20 to 60 microns, and much coarser powders having an average particles size of 60 to 500 microns; the finer powders, the coarser powders, and the much coarser powders being different substances. For example, the finer powders have an average particle size of <5 to 15 microns, the coarser powders have an average particles size of 30 to 50 microns, and the much coarser powders have an average particles size of 70 to 90 microns. The proportion of finer excipient powders may be 0.1% to 99% of the total amount of excipient powders.

In some embodiments, where the pharmaceutically acceptable excipient of the dry powder formulations is a mixture of finer powders and coarser powders, the powdered palonosetron may be blended with the finer excipient powders first, and then the mixture of the powdered palonosetron and the finer powders are blended with the coarser excipient powders. Alternatively, the powdered palonosetron may be blended with the finer excipient powders and the coarser excipient powders separately, and then each of the blended mixtures (i.e., finer excipient powders with powdered palonosetron and coarser excipient powders with powdered palonosetron) are blended with each other. Alternatively, the finer excipient powders and the coarser excipient powders are blended first, and then the blended excipient mixture is further blended with powdered palonosetron.

In some embodiments, where the pharmaceutically acceptable excipient of the dry powder formulations is a mixture of finer powders, coarser powders, and much coarser, the powdered palonosetron may be sequentially blended with the finer excipient powders, the coarser excipient powders, and the much coarser excipient powders. Alternatively, the powdered palonosetron is blended with the finer excipient powders, the coarser excipient powders, and the much coarser excipient powders separately, and then the mixtures (i.e., finer excipient powders with powdered palonosetron, coarser excipient powders with powdered palonosetron, and much coarser excipient powders with palonosetron) are blended with each other. Alternatively, the finer excipient powders, the coarser excipient powders, and the much coarser excipient powders are blended first, and then the blended excipient mixture is further blended with powdered palonosetron.

In some embodiments, the content of the powdered palonosetron in the dry powder formulations ranges from 0.01% to about 100% of the total composition of formulation, for example from about 0.01% to about 50%, from about 0.01% to about 45%, from about 0.01% to about 40%, from about 0.01% to about 35%, from about 0.01% to about 30%, from about 0.01% to about 25%, from about 0.01% to about 20%, from about 0.01% to about 15%, or from about 0.01% to about 10% of the total composition of formulation.

In some embodiments, the content of the powdered palonosetron in the dry powder formulations may also range from about 0.1% to about 100%, from about 0.1% to about 50%, from about 0.1% to about 45%, from about 0.1% to about 40%, from about 0.1% to about 35%, from about 0.1% to about 30%, from about 0.1% to about 25% of the total composition of formulation, from about 0.1% to about 20%, from about 0.1% to about 15%, or from about 0.1% to about 10% of the total composition of formulation, for example from about 1% to about 10% of the total composition of formulation, and for example from about 5% to about 10% of the total composition of formulation. In some embodiments, with respect to the formulations, the powdered palonosetron is about: 10%, 9%, 8%, 7%, 6%, 5%, 4%, 3%, 2%, 1%, or 0.5% of the total composition of formulation.

In some embodiments, the dry powder formulations contain 0.01-10 mg of the powdered palonosetron, for example from 0.05-5 mg, from 0.1-1 mg, from 0.25-0.75 mg, or about 0.5 mg.

In some embodiments, the dry powder formulations comprise palonosetron and lactose. The dry powder formulations containing lactose comprise palonosetron, finer lactose, and coarser lactose, or palonosetron, finer lactose, and much coarser lactose palonosetron, or finer lactose, coarser lactose, and much coarser lactose. For example, the dry powder formulations may comprise about 0.1 to about 1 mg of palonosetron, about 0.001 to about 2.5 g of finer lactose, and about 0.001 to about 2.5 g of coarser lactose. For example, in dry powder formulations containing palonosetron and lactose or glucose, the amount of the palonosetron is from 0.1-1 mg, and the amount of lactose or glucose is about 0.001 g to about 2.5 g. For example, the amount of palonosetron is about 0.1 to about 1 mg and the amount of lactose or glucose is about 1 to about 200 mg.

In some embodiments, the dry powder formulations comprise palonosetron and glucose. The dry powder formulations containing glucose comprise palonosetron, finer glucose, and coarser glucose, or palonosetron, finer glucose, coarser glucose, and much coarser glucose. In some embodiments, the dry powder formulations comprise about 0.1 to about 1 mg of palonosetron, about 0.001 to about 2.5 g of finer glucose, and about 0.001 to about 2.5 g of coarser glucose. For example, the dry powder formulations may comprise about 0.1 to about 1 mg of palonosetron, about 1 to about 200 mg of finer glucose, and about 1 to about 200 mg of coarser glucose.

In some embodiments, the dry powder formulations comprise palonosetron, lactose, and glucose. The dry powder formulations comprising palonosetron, lactose, and glucose may comprise palonosetron, finer lactose, and coarser glucose or palonosetron, finer glucose, and coarser lactose. For example, the dry powder formulations may comprise about 0.1 to about 1 mg of palonosetron, from about 0.001 to about 2.5 g of lactose, and from about 0.001 to about 2.5 g of glucose. In some embodiments, the dry powder formulations comprise from about 0.1 to about 1 mg palonosetron, from about 0.001 to 2.5 g of finer lactose, and from about 0.001 to about 2.5 g of coarser glucose. In an alternative embodiment, the formulation comprises from about 0.1 to about 1 mg of palonosetron, from about 0.001 to about 2.5 g of finer glucose, and from about 0.001 to about 2.5 g of coarser lactose.

In some embodiments, the aerosol formulations described herein are uniform and homogeneous. The uniformity/homogeneity of the aerosol formulations is measured by drawing 3 or more samples from the formulation, dissolving in mobile, and testing for concentration of the active pharmaceutical ingredient (API, palonosetron) in the formulation by HPLC. The uniformity of the aerosol formulations is expressed by the relative standard deviation (% RSD) of the API concentration. The aerosol formulations have an RSD % less than 5%, less than 4%, less than 3%, less than 2.5%, less than 2.25%, less than 2.0%, less than 1.75%, less than 1.5%, less than 1.25%, less than 1.0%, less than 0.75%, less than 0.5%, less than 0.25%, and even less than 0.1%.

In some embodiments, the discharge capacity or percent recovery of the aerosol formulations is measurable with a Next Generation Pharmaceutical Impactor (NGI). In this device, powders are drawn by vacuum into different chambers representing the lung, each chamber corresponding to a different range of aerodynamic particle size. NGI data includes mass median aerodynamic diameter (MMAD), and fine particle fraction (FPF). The discharge capacity or percent recovery of the aerosol formulations described herein is at least 70%, at least 75%, at least 80%, at least 85%, at least 90%, at least 91%, at least 92%, at least 93%, at least 94%, at least 95%, at least 96%, at least 97%, at least 98%, and even at least 99%, as measured by NGI.

In some embodiments, the inclusion of fine excipient particles increases the FPF while decreasing the MMAD.

In some embodiments, exemplary formulations containing coarse and fine lactose particles are expected to achieve a 3-10% increase in the delivery of FPF when the humidity of the environment during the aerodynamic performance testing is to be controlled to have a relative humidity (RH) of 50% rather than the ambient 20% RH. It is believed that the higher-than-ambient humidity, which is more representative of the environment in the human inhalation route, further increases the disaggregation by reducing the surface-energy-induced-aggregation when the formulation is inhaled into the impactor.

In some embodiments, the dry powdered formulations are administered by a dry powder inhaler, a dry powder dispenser, or a dry powder delivery device. The inhaler may be a single dose or multi-dose inhaler. Suitable inhalers may include SPINHALER®, ROTAHALER®, AEROLIZER®, RS01®, INHALATOR®, HANDIHALER®, DISKHALER®, DISKUS®, ACCUHALER®, AEROHALER®, ECLIPSE®, TURBOHALER®, TURBUHALER®, EASYHALER®, NOVOLIZER®, CLICKHALER®, PULVINAL®, NEOHALER®, SKYEHALER®, XCELOVAIR®, PULVINA®, TAIFUN®, MAGHALER®, TWISTHALER®, JETHALER®, FLOWCAPS®, XCAPS®, TWINCAPS®, CYCLOHALER®, TURBOSPIN®, AIR DPI®, ORBITAL®, DIRECTHALER®, or an inhaler that is newly developed, or other appropriate devices.

Pressurized Metered Dose Formulations (pMDI Formulations)

In some embodiments, the formulation is a pMDI formulation containing an excipient, where the excipient selected from the group consisting of oleates, stearates, myristates, alkylethers, alkyl arylethers, sorbates, and mixtures thereof. In the pMDI formulations, the excipient may include sorbitan trioleate, isopropyl myristate, or lecithin. Additional excipients for the pMDI formulations include oleic acid or oleic acid esters and polyvinylpyrrolidone (PVP).

In some embodiments, the pMDI formulations do not include a propellant. However, the pMDI formulations generally include a propellant, especially a hydrofluoroalkane propellant. The hydrofluoroalkane propellants for the pMDI formulations are selected from the group consisting of 1,1,1,2-tetrafluoroethane (P134a), 1,1,1,2,3,3,3-heptafluoro-n propane (P227), and mixtures P134a and P227. Another suitable propellant for the pMDI formulations is 2H, 3H-perfluoropentane (HPFP).

In some embodiments, the pMDI formulations may include a diluent or a mixture of diluents. The pMDI formulations may also include a surfactant or a mixture of surfactants. Exemplary surfactants are selected from the group consisting of alkylethers, alkyl arylethers, laurates, myristates, oleates, sorbates, stearates, propylene glycol, lipids, and combinations thereof. Exemplary surfactants are oleates, sorbates, stearates, propylene glycol, and combinations thereof.

In some embodiments, the pMDI formulations do not comprise a co-solvent. However, in alternate embodiments, the pMDI formulations contain a co-solvent or a mixture of co-solvents. The pMDI formulations may include a co-solvent selected from C₂₋₆ alcohols, polyols, cineole, citral, lactic acid oligomers, or poly(ethylene glycols).

In some embodiments, the pMDI formulations may comprises ethanol as a co-solvent. The content of ethanol in the pMDI formulations is no more than 25% (w/w), no more than 20% (w/w), no more than 15% (w/w), no more than 10% (w/w), no more than 8% (w/w), for example no more than 5% (w/w) of ethanol, no more than 2.5% (w/w), and for example no more than 1% (w/w) of ethanol.

In some embodiments, the palonosetron in the pMDI formulations is present from about 0.01% to about 20%, from about 0.01% to about 10%, from 0.01% to about 1%, from about 0.01% to about 0.1%, from about 0.01% to about 0.05%, or from about 0.01% to about 0.025% of the total composition of the formulation. In some embodiments, with respect to the pMDI formulations, the content of the palonosetron is from about 0.025% to about 0.05% of the total composition of the formulation.

In some embodiments, the pMDI formulations comprise palonosetron and at least one selected from P134a and P227. In such pMDI formulations, based on the size of the canister, the amount of palonosetron is from 0.01-25 mg and the amount of P134a and/or P227 is about 0.5 g to about 50 g. In some embodiments, the pMDI formulation contains palonosetron and P134a, where the amount of palonosetron is from about 0.01 to about 25 mg, and the amount of P134a is from 0.5 g to about 50 g, for example about 10 g to 20 g. Similarly, the pMDI formulation may contains palonosetron and P227, where the amount of palonosetron is from about 0.01 to about 25 mg and the amount of P227 is from 0.5 g to about 50 g, for example about 10 g to 20 g. In pMDI formulations containing palonosetron, P134a, and P227, the amount of palonosetron is from about 0.01 to about 25 mg and the amount of P134a about 0.5 g to about 50 g, and the amount of P227 is about 0.5 g to about 50 g.

In some embodiments, the pMDI formulation comprises palonosetron, P134a and/or P227, and isopropyl myristate. In some embodiments, the pMDI formulations contain palonosetron, P134a and/or P227, and propylene glycol. In addition, the pMDI formulations may contain palonosetron, P134a and/or P227, and isopropyl laurate.

In some embodiments, the pMDI formulations described herein contain palonosetron that is a solute in a solution, and the solvent is selected from a group consisting of propellants, hybrid propellants, cosolvents, cosolvent mixture, organic solvents, water, buffers, and combinations thereof.

In some embodiments, the pMDI formulations described herein contain palonosetron that is in a powdered form in a suspension, and the suspension is in a liquid selected from a group consisting of propellants, hybrid propellants, propellants with stabilizers, propellants with surfactants, propellants with diluents, propellants with cosolvents, water, buffer, and combinations thereof.

In some embodiments, the pMDI formulations described herein contain palonosetron that is a solute in a solution, wherein the solubility of palonosetron is more than 0.01% w/w, more than 0.1% w/w, or more than 1%.

In some embodiments, the pMDI formulations described herein contain palonosetron that is in a powdered form in a suspension, wherein the solubility of palonosetron is less than 0.1% w/w, less than 0.01% w/w, less than 0.001%, or less than 0.0002% w/w.

In some embodiments, the pMDI formulations are administered by an actuator, a metered dose inhaler, an aerosol dispenser, or an aerosol delivery device.

The present disclosure also provides methods of treating a condition of nausea or vomiting, wherein the method comprises pulmonary administration of a pharmaceutically acceptable amount of the aerosol formulations described herein; and wherein the formulations are administered into the pulmonary tract by inhalation. In some embodiments, the pulmonary delivery of palonosetron to a subject is carried out by having the subject inhale a pharmaceutically acceptable amount of the aerosol formulation described herein through the subject's mouth. In some embodiments, the pulmonary delivery of palonosetron to a subject is accomplished by having the subject inhale a pharmaceutically acceptable amount of the aerosol formulation described herein through the subject's nose.

In some embodiments, the pharmaceutically acceptable amount is produced by introducing the palonosetron into a gas stream. Specifically, the pharmaceutically acceptable amount is produced by introducing the palonosetron into a gas stream, and the gas stream is the subject's inspiratory breath.

In some embodiments, with respect to the methods, the pharmaceutically acceptable amount contains about 0.01 mg to about 25 mg of palonosetron and the total dosage is from about 0.01 mg to about 25 mg.

In some embodiments, the pharmaceutically acceptable amount contains less than about 25 mg, less than about 10 mg, less than about 1 mg, less than about 0.5 mg, less than about 0.25 mg, or less than about 0.1 mg of palonosetron. In some embodiments, the pharmaceutically acceptable amount contains more than about 0.01 mg, more than about 0.25 mg, more than about 0.5 mg, more than about 1 mg, or more than about 10 mg of palonosetron. For example, the pharmaceutically acceptable amount contains about 0.5 mg of palonosetron.

In some embodiments, the total dosage of palonosetron per day is about 0.01 mg to about 25 mg, about 0.1 mg to about 10 mg, about 0.25 mg to about 1 mg, about 0.5 mg of palonosetron per day. In some embodiments, a dosage can be administered once in its entirety or in divided dosages for example 2, 3, 4, or 5 dosages per day.

With the dry powder formulations, the pharmaceutically acceptable amount of palonosetron is produced by releasing blended powders containing powdered palonosetron from a container such as a capsule or a blister by using a device such as a dry powder inhaler. A device may be loaded with one or more capsules/blisters at a time. The pharmaceutically acceptable amount is produced through one, two or multiple actuations. The releasing amount of one actuation is for example equal to the formulation stored in one capsule or blister. Whereas with the pMDI formulations, the pharmaceutically acceptable amount of palonosetron is produced by releasing a propellant containing palonosetron from a container such as a canister by using a device such as a pMDI inhaler. The canister may be actuated by pressing an actuator or by inhalation. The pharmaceutically acceptable amount is produced through one, two or multiple actuations. The releasing amount of one actuation is for example less than the formulation stored in one canister. The releasing amount is metered.

In some embodiments, after administration to a subject, palonosetron in blood plasma reaches a maximum concentration (Cmax) of 1-5000 ng/mL in the subject, for example of 2-2000 ng/mL, and for example of 5-1000 ng/mL in a subject.

In some embodiments, delivery of the aerosol formulations through the pulmonary tract of a subject provides a Cmax of palonosetron in blood plasma that is about 0.05 to about 1, about 0.1 to about 0.8, about 0.2 to about 0.6, or about 0.3 to about 0.4 times of the Cmax achieved following intravenous bolus delivery of palonosetron. Moreover, delivery of the aerosol formulations through the pulmonary tract of a subject to provides a Cmax of palonosetron in blood plasma that is about 0.1 to about 1.5, about 0.2 to about 1.25, about 0.4 to about 1.1, or about 0.8 to about 1.05 times of the Cmax achieved following oral delivery of palonosetron.

In some embodiments, the palonosetron in blood plasma reaches maximum concentration at (Tmax) 1 minute to 2 hours after dose in a subject, for example the Tmax is 2 minutes to 1 hour after dose in a subject, and even 5 minutes to 30 minutes after dose in a subject. Delivery of the aerosol formulations through the pulmonary tract of a subject provides a Tmax of palonosetron in blood plasma that is about 0.01 to about 1.5, about 0.05 to about 1, about 0.1 to about 0.8, about 0.2 to about 0.6, or about 0.3 to about 0.4 times of the Tmax achieved following oral delivery of palonosetron.

In some embodiments, the area under curve (AUC) of palonosetron in blood plasma of a subject ranges from 2-50000 ng*h/mL, for example from 5-20000 ng*h/mL, and for example from 10-10000 ng*h/mL. Delivery of the aerosol formulations through the pulmonary tract produces a mean AUC of palonosetron in blood plasma that is about 0.1 to 1.5, about 0.2 to about 1.25, about 0.4 to about 1.1, or about 0.8 to about 1.05 times of the mean AUC achieved following intravenous bolus delivery of palonosetron. In some embodiments, the AUC is about the same as that is achieved following intravenous bolus delivery of palonosetron. Similarly, delivery of the aerosol formulations through the pulmonary tract produces a mean total AUC of palonosetron in blood plasma that is about 0.1 to about 1.5, about 0.2 to about 1.25, about 0.4 to about 1.1, or about 0.8 to about 1.05 times of the AUC achieved following oral delivery of palonosetron. In some embodiments, the AUC is about the same as that is achieved following oral delivery of palonosetron.

In some embodiments, the aerosol and dry powder formulations and the method are useful for the reduction, elimination, or prevention of nausea and vomiting, where the nausea and vomiting are chemotherapy-induced nausea and vomiting, radiation-induced nausea, or vomiting and post-operative nausea and vomiting.

In some embodiments, the subject is a cancer patient; for example a cancer patient undergoing chemotherapy, radiotherapy, or a surgery. Additionally, the cancer patient may suffer from nausea and/or vomiting related to the chemotherapy, radiotherapy, or surgery.

In some embodiments, the powdered palonosetron of the aerosol formulations may be prepared by dissolving the bulk palonosetron in distilled water with co-solvents, to form a solution; spray drying the solution, to obtain powdered palonosetron; separating and filtering the powdered palonosetron according to their sizes with a cyclone; milling and grinding the powdered palonosetron to further reduce the size of powdered palonosetron; and collecting and analyzing the precipitated palonosetron powder. During the milling and grinding, the milling and grinding forces and timing are optimized so that the particle size distribution of the processed palonosetron is from about 0.5 to about 5 μm; and the mean volume diameter is of about 2-3 μm.

In some embodiments, the powdered palonosetron of the aerosol and dry powder formulations may also be prepared by dissolving the bulk palonosetron in distilled water, to form a solution; spray drying the solution with temperature in a drying vessel; separating and filtering the powdered palonosetron according to their sizes with a cyclone; and collecting and analyzing the precipitated palonosetron powder. The flow rate of the solution, the temperature and the flow rate of the drying air, and other parameters are optimized so that the palonosetron precipitation is crystalline; and the particle size distribution is of about 0.5 to about 5 μm; and the mean volume diameter is of about 2-3 μm.

In some embodiments, the powdered palonosetron of the aerosol and dry powder formulations may be prepared by dissolving the bulk palonosetron in supercritical fluid CO₂, to form a solution; depressuring the solution in a depressurization vessel; and collecting and analyzing the precipitated palonosetron powder. The temperature and the pressure of the SCF CO₂ (before the precipitation) and the depressurization vessel, and other parameters are optimized so that the palonosetron precipitation is crystalline; and the particle size distribution is of about 0.5 to about 5 μm; and the mean volume diameter is of about 2-3 μm.

In some embodiments, for dry powder formulations, the powdered palonosetron may be mixed with one or more excipients, to form the dry powder formulation. The obtained dry powder formulation is then loaded into a dry powder inhaler. Alternatively, for pMDI formulations, the palonosetron may be mixed with a pressurized propellant or mixture of propellants, to form the pMDI formulation. The obtained pMDI formulation is then filled into canisters, which are installed into a metered-dose inhaler.

In some embodiments, the present disclosure also provides pharmaceutical aerosol inhalation formulations or inhalable pharmaceutical aerosol formulations for pulmonary administration to a subject, wherein
the formulation is a dry power formulation and comprises powdered palonosetron;
the powdered palonosetron is produced by a particle engineering process;
the MMAD of powdered palonosetron is between 1 and 3 microns;
the formulation may comprise excipient(s);
the formulation is administered into the pulmonary tract by inhalation; and/or
the subject is a cancer patient suffering from nausea that is related to chemotherapy, radiotherapy, or surgery;
or the formulation is a pMDI formulation comprises palonosetron;
the palonosetron may be powdered palonosetron produced by a particle engineering process;
the MMAD of powdered palonosetron is between 1 and 3 microns;
the formulation may comprise excipient(s) and at least a hydrofluoroalkane;
the formulation is administered into the pulmonary tract by inhalation; and/or
the subject is a cancer patient suffering from nausea that is related to chemotherapy, radiotherapy, or surgery.

In some embodiments, the powdered palonosetron may be produced by a spray drying process that comprises:
i) dissolving the bulk palonosetron in distilled water, to form a solution;
ii) spray drying the solution in a spray dryer;
iii) separating and filtering the palonosetron particles according to their sizes with a cyclone; and/or
iv) collecting and analyzing the precipitated palonosetron powder.

In some embodiments, the powdered palonosetron is produced by a supercritical fluid process that comprises:
i) dissolving the bulk palonosetron in supercritical fluid CO₂, to form a solution;
ii) depressuring the saturated solution in a depressurization vessel; and/or
iii) collecting and analyzing the precipitated palonosetron powder.

In some embodiments, the formulation is a pharmaceutical dry powder inhalation formulation that contains lactose and/or glucose as an excipient, where the amount of palonosetron is about 0.01 to 100 wt %, about 1 to 50 wt %, about 2 to 20 wt %, or about 5 to 15 wt % of the excipient. In some embodiments, the formulation is a pharmaceutical pMDI inhalation formulation that contains P134a and/or P227 as propellants, where the amount of palonosetron is about 0.01 to 20 wt %, about 0.01 to 1 wt %, or about 0.01 to 0.5 wt % of the propellant.

In some embodiments, delivery of the pharmaceutical aerosol inhalation formulations into the pulmonary tract of a subject provides a Cmax of palonosetron in blood plasma that is about 20-80% of the Cmax achieved following intravenous bolus delivery of palonosetron. The Cmax from delivery into the pulmonary tract may be about the same as the Cmax achieved following oral delivery of palonosetron.

In some embodiments, pulmonary delivery of the formulations described herein to a subject provides a Cmax of palonosetron (e.g., in blood plasma) that is about: >1 times, >1.5 times, >2 times, >2.5 times, >3 times, >3.5 times, >4 times, >4.5 times, or >5 times, of the Cmax achieved following oral delivery of palonosetron.

In some embodiments, pulmonary delivery of the formulations described herein to a subject provides a Cmax of palonosetron (e.g., in blood plasma) that is about: the same <1.5 times, <2 times, <2.5 times, <3 times, <3.5 times, <4 times, <4.5 times, or <5 times, of the Cmax achieved following intravenous delivery of palonosetron.

In some embodiments, delivery of the pharmaceutical aerosol inhalation formulations into the pulmonary tract of a subject provides Tmax of palonosetron in blood plasma that is less than the Tmax achieved following oral delivery of palonosetron.

In some embodiments, pulmonary delivery of the formulations described herein to a subject provides a Tmax of palonosetron (e.g., in blood plasma) that is about: <0.1 times, <0.2 times, <0.3 times, <0.4 times, <0.5 times, <0.6 times, <0.7 times, <0.8 times, or <1 times of the Tmax achieved following oral delivery of palonosetron. In some embodiments, pulmonary delivery of the formulations described herein to a subject provides a Tmax of palonosetron (e.g., in blood plasma) that is about the same as the Tmax achieved following intravenous delivery of palonosetron.

In some embodiments, delivery of the pharmaceutical aerosol inhalation formulations into the pulmonary tract of a subject provides also provides an AUC of palonosetron in blood plasma that is about the same as the AUC achieved following intravenous bolus delivery of palonosetron.

In some embodiments, pulmonary delivery of the formulations described herein to a subject provides an AUC of palonosetron (e.g., in blood plasma) that is about: >1 times, >1.5 times, >2 times, >2.5 times, >3 times, >3.5 times, >4 times, >4.5 times, or >5 times, of the AUC achieved following oral delivery of palonosetron.

Additional embodiments within the scope provided herein are set forth in non-limiting fashion elsewhere herein and in the examples. It should be understood that these examples are for illustrative purposes only and are not to be construed as limiting in any manner.

### Pulmonary Aerosol Formulations

As described herein, the aerosol formulations described herein comprise palonosetron and the formulations are useful for pulmonary delivery via inhalation. In some embodiments, the active drug palonosetron when administered by inhalation penetrate deep into the lungs in order to show physiological action. In order to achieve this, the palonosetron inhaled should be in the powdered form. For example, the MMAD of palonosetron drug does not exceed about 5 μm.

### Powdered Palonosetron

The powdered palonosetron can be prepared by processes of micronization, such as mechanical grinding, attrition by jet milling, solution precipitation, spray drying, lyophilization, and supercritical fluid processes.

Spray dying followed by a cyclone separation/filtering process may produce respirable particles rapidly and efficiently.

Direct controlled crystallization using an antisolvent precipitation technique may produce respirable particles with expected shapes. The particle size may be controlled by using one or more growth-retarding stabilizing additives.

Supercritical fluid processes may be used to produce respirable particles of the desired size. The supercritical processes may be used to prepare powdered palonosetron may include rapid expansion, solution enhanced diffusion, gas-anti solvent, supercritical antisolvent, precipitation from gas-saturated solution, precipitation with compressed antisolvent, aerosol solvent extraction system, or combinations of the foregoing. For example, the process can be rapid expansion of supercritical solution (RESS).

The powdered palonosetron prepared by the above processes may have an MMAD between 0.5 and 5 μm.

The amount of the powdered palonosetron in the formulation may be about 0.01% to about 100% of the total composition of formulation. For example, the amount of the powdered palonosetron may be 0.01% to about 20% of the total composition of formulation.

### Dry Powder Formulations

Excipients

The dry powder formulations described herein may comprise pharmaceutically acceptable excipients. The excipients which may be used in the formulation include carbohydrates, amino acids, polypeptides, lipids, antioxidants, salts, polyalcohols, galactose, mannose, sorbose, lactose, glucose, trehalose, raffinose, maltodextrins, dextrans, mannitol, xylitol, alanine, glycine, tryptophan, tyrosine, leucine, phenylalanine, polyvinylpyrrolidone (PVP), 2,6-di-tert-butyl-p-cresol (DBPC), and mixtures or combinations thereof.

pMDI Formulations

Propellants

The pMDI formulations described herein may comprise pharmaceutically acceptable propellants. Propellants can include hydrofluoroalkane (HFA) propellants. The hydrofluoroalkane propellants which may be used in the pMDI formulations include 1,1,1,2-tetrafluoroethane (P134a), 1,1,1,2,3,3,3-heptafluoro-n propane (P227), and mixtures of P134a and P227.

### Excipients

The pMDI formulations described herein may comprise pharmaceutically acceptable excipients. Excipients can include carbohydrates, amino acids, polypeptides, lipids, antioxidants, salts, polyalcohols, galactose, mannose, sorbose, lactose, glucose, trehalose, raffinose, maltodextrins, dextrans, mannitol, xylitol, alanine, glycine, tryptophan, tyrosine, leucine, phenylalanine, oleates, stearates, myristates, alkylethers, alkyl arylethers, sorbates, esters, polyvinylpyrrolidone (PVP), 2,6-di-tert-butyl-p-cresol (DBPC), and mixtures or combinations thereof.

### Surfactants

The pMDI formulations described herein may comprise pharmaceutically acceptable surfactants. Surfactants can include alkylethers, alkyl arylethers, laurates, myristates, oleates, sorbates, stearates, propylene glycol, lipids, and combinations thereof.

### Co-Solvents

The pMDI formulations described herein may comprise pharmaceutically acceptable co-solvents. Co-solvents can include C₂₋₆ alcohols, polyols, and combinations thereof. For example the co-solvent may be ethanol.

### Exemplary Formulations

The following examples illustrate some embodiments of the disclosure and are not intended to be construed in a limiting manner.

Formulation 1

| Ingredient | Amount |
|---|---|
| Palonosetron Fine Powder | 0.5 mg |

Formulation 2

| Ingredient | Amount |
|---|---|
| Palonosetron Fine Powder | 0.5 mg |
| Lactose Powder | 4.5 mg |

Formulation 3

| Ingredient | Amount |
|---|---|
| Palonosetron Fine Powder | 0.5 mg |
| Glucose Powder | 4.5 mg |

Formulation 4

| Ingredient | Amount |
|---|---|
| Palonosetron Fine Powder | 0.25 mg |
| Finer Lactose Powder | 0.225 mg |
| Coarser Lactose Powder | 2.25 |

Formulation 5

| Ingredient | Amount |
|---|---|
| Palonosetron Fine Powder | 0.25 mg |
| Finer Lactose Powder | 2.5 mg |
| Coarser Lactose Powder | 10 mg |

Formulation 6

| Ingredient | Amount |
|---|---|
| Palonosetron Fine Powder | 0.075 mg |
| Finer Lactose Powder | 0.5 mg |
| Coarser Lactose Powder | 7 mg |

Formulation 7

| Ingredient | Amount |
|---|---|
| Palonosetron Fine Powder | 0.5 mg |
| Finer Lactose Powder | 0.45 mg |
| Coarser Lactose Powder | 4.05 mg |

Formulation 8

| Ingredient | Amount |
|---|---|
| Palonosetron Fine Powder | 0.075 mg |
| Finer Lactose Powder | 1.25 mg |
| Coarser Lactose Powder | 11.25 mg |

Formulation 9

| Ingredient | Amount |
|---|---|
| Palonosetron Fine Powder | 0.5 mg |
| Finer Lactose Powder | 3.75 mg |
| Coarser Lactose Powder | 8.75 mg |

Formulation 10

| Ingredient | Amount |
|---|---|
| Palonosetron Fine Powder | 0.25 mg |
| Glucose Powder | 2.25 mg |

Formulation 11

| Ingredient | Amount |
|---|---|
| Palonosetron Fine Powder | 0.5 mg |
| Finer Glucose Powder | 0.45 mg |
| Coarser Glucose Powder | 4.05 mg |

Formulation 12

| Ingredient | Amount |
|---|---|
| Palonosetron Fine Powder | 0.075 mg |
| Finer Glucose Powder | 2.5 mg |
| Coarser Glucose Powder | 10 mg |

Formulation 13

| Ingredient | Amount |
|---|---|
| Palonosetron Fine Powder | 0.25 mg |
| Lactose Powder | 1.125 mg |
| Glucose Powder | 1.125 mg |

Formulation 14

| Ingredient | Amount |
|---|---|
| Palonosetron Fine Powder | 0.25 mg |
| Dipalmitoylphosphatidylcholine (DPPC) | 0.025 mg |

Formulation 15

| Ingredient | Amount |
|---|---|
| Palonosetron | 5 mg |
| HFA 134a Propellant | 10 g |

Formulation 16

| Ingredient | Amount |
|---|---|
| Palonosetron | 5 mg |
| HFA 134a Propellant | 10 g |
| Isopropyl Myristate | 0.1 g |

Formulation 17

| Ingredient | Amount |
|---|---|
| Palonosetron | 5 mg |
| HFA 227 Propellant | 10 g |

Formulation 18

| Ingredient | Amount |
|---|---|
| Palonosetron | 5 mg |
| HFA 227 Propellant | 10 g |
| Isopropyl Myristate | 0.1 g |

Formulation 19

| Ingredient | Amount |
|---|---|
| Palonosetron | 5 mg |
| HFA 134a Propellant | 20 g |

Formulation 20

| Ingredient | Amount |
|---|---|
| Palonosetron | 5 mg |
| HFA 227 Propellant | 20 g |

Formulation 21

| Ingredient | Amount |
|---|---|
| Palonosetron | 5 mg |
| HFA 134a Propellant | 10 g |
| HFA 227 Propellant | 10 g |

Formulation 22

| Ingredient | Amount |
|---|---|
| Palonosetron | 5 mg |
| HFA 134a Propellant | 10 g |
| HFA 227 Propellant | 10 g |
| Isopropyl Laurate | 0.1 g |

Formulation 23

| Ingredient | Amount |
|---|---|
| Palonosetron | 0.5 mg |
| HFA 134a Propellant | 1 g |

Formulation 24

| Ingredient | Amount |
|---|---|
| Palonosetron | 0.5 mg |
| HFA 134a Propellant | 1 g |
| Isopropyl Myristate | 0.01 g |

Formulation 25

| Ingredient | Amount |
|---|---|
| Palonosetron | 0.5 mg |
| HFA 227 Propellant | 1 g |

Formulation 26

| Ingredient | Amount |
|---|---|
| Palonosetron | 0.5 mg |
| HFA 227 Propellant | 1 g |
| Isopropyl Myristate | 0.01 g |

Formulation 27

| Ingredient | Amount |
|---|---|
| Palonosetron | 0.5 mg |
| HFA 134a Propellant | 2 g |

Formulation 28

| Ingredient | Amount |
|---|---|
| Palonosetron | 0.5 mg |
| HFA 227 Propellant | 2 g |

Formulation 29

| Ingredient | Amount |
|---|---|
| Palonosetron | 0.5 mg |
| HFA 134a Propellant | 1 g |
| HFA 227 Propellant | 1 g |

Formulation 30

| Ingredient | Amount |
|---|---|
| Palonosetron | 0.5 mg |
| HFA 134a Propellant | 1 g |
| HFA 227 Propellant | 1 g |
| Isopropyl Laurate | 0.01 g |

Formulation 31

| Ingredient | Amount |
|---|---|
| Palonosetron | 0.5 mg |
| Poly(vinyl alcohol) (PVA) | 0.25 mg |

### Example 1: Preparation of Spray Dried Palonosetron Fine Powder

Powdered palonosetron was prepared by spray drying with SPRAY DRYER SD-MICRO™ (manufactured by GEA Process Engineering, Inc., Columbia, Md., USA). The experiments were done at GEA Process Engineering, Inc., Columbia, Md., USA.

### Example 2: Particle Size Distribution of Spray Dried Palonosetron Fine Powder

The Particle Size Distribution of the Palonosetron Fine Powder, prepared by Spray Drying using the above parameters, was measured by Malvern Mastersizer (Malvern Instruments, UK) at GEA Process Engineering, Inc., Columbia, Md., USA.

### Example 3: Preparation of Jet Milled Palonosetron Fine Powder

Palonosetron HCl was milled with a 2-in pancake Jet Mill and flexible containment. The process air was nitrogen. The jet milling was conducted at Catalent Micron Technologies, Malvern, Pa., USA.

### Example 4: Particle Size Distribution of Jet Milled Palonosetron Fine Powder

The particle size distribution of the palonosetron fine powder, prepared by jet milling, was measured by HELOS Particle Size Analyzer (Sympatec GmbH, Germany) at Micron Technologies, Inc., Malvern, Pa., USA.

The Particle Size Distribution parameters are:
D10: 0.68 μm
D50: 1.39 μm
D90: 2.89 μm
D95: 3.68 μm
D99.5: 6.92 μm
Cumulative %<9 μm: 100%
SMD: 1.20 μm
VMD: 1.65 μm

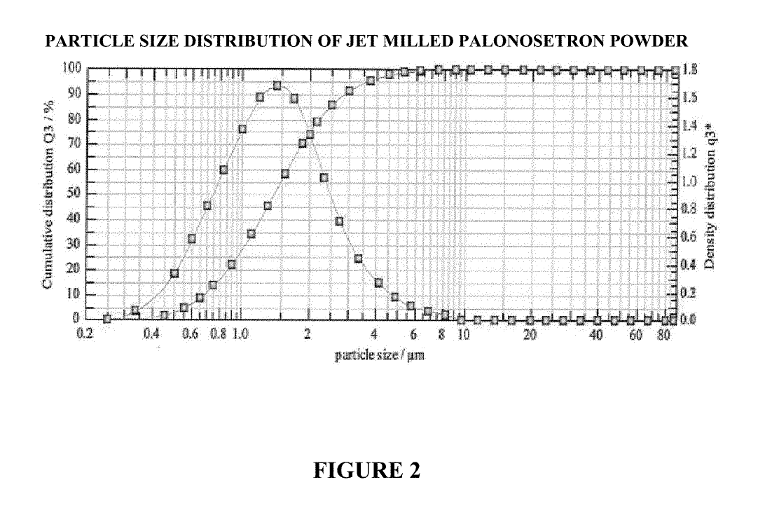

FIG. 2 shows an exemplary Particle Size Distribution of the Jet Milled Palonosetron.

### Example 5: Blending Uniformity

Each of the exemplary formulation blends were produced with a TURBULA® Shaker Mixer. If there were fine and coarse lactose, the coarse lactose and the fine lactose were blended together before the addition of the micronized palonosetron HCl. All blending was performed at 48 rpm for 2 cycles of 15 minutes. After a blending cycle was complete, the contents were passed through a 300 μm aperture sieve. All blends produced were tested for Batch Uniformity/Potency. Batch Uniformity/Potency was tested by drawing 5 samples from each blend, dissolving in mobile phase, and testing for API concentration by HPLC. Test results for batch uniformity were measured as % relative standard deviation (% RSD) of the 5 potency measurements. Blend uniformity test results are presented in Table 1 below for 3 Exemplary Formulations (EF-I, EF-II and EF-III) described in the Exemplary Formulations Section.

TABLE 1
Blend Uniformity Testing

| | EF-I | EF-II | EF-III |
|---|---|---|---|
| % RSD | 3.0 | 1.2 | 2.0 |

### Example 6: Aerodynamic Performance by Next Generation Impaction

The in vitro aerodynamic performance of the dry powder palonosetron aerosol formulations, including fine particle fraction (FPF), Mass Median Aerodynamic Diameter (MMAD) and Geometric Standard Deviation (GSD), were tested by Next Generation Impaction (NGI) at Drug Dynamics Institute, College of Pharmacy, The University of Texas at Austin, Austin, Tex., USA. The results reflect the in vivo (pulmonary) aerodynamic performance of the above exemplary aerosol formulations. The Next Generation Impactor used in this embodiment is made by Copley Scientific, GB.

RS01® was used as the model Dry Powder Inhaler Device. The flow rate was 60 LPM (>4 kPa), the duration was 4 seconds; the total volume was 4 L. All 3 exemplary aerosol formulations (EF-I, EF-II, and EF-III) were tested in triplicates (n=3) at controlled environmental conditions of 23.6° C. and 46% RH.

NGI testing was conducted in accordance with the USP 36<601>, Apparatus 5. Extraction was conducted for the capsule, device, device adaptor, throat, pre-separator, stages 1 through 7, and MOC with a sufficient quantity of sample diluent. Device adaptor and device were extracted together and are reported as “device”.

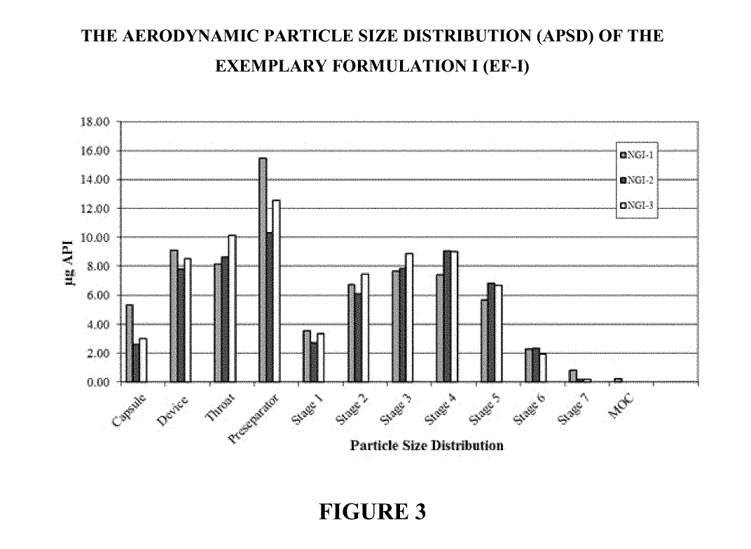

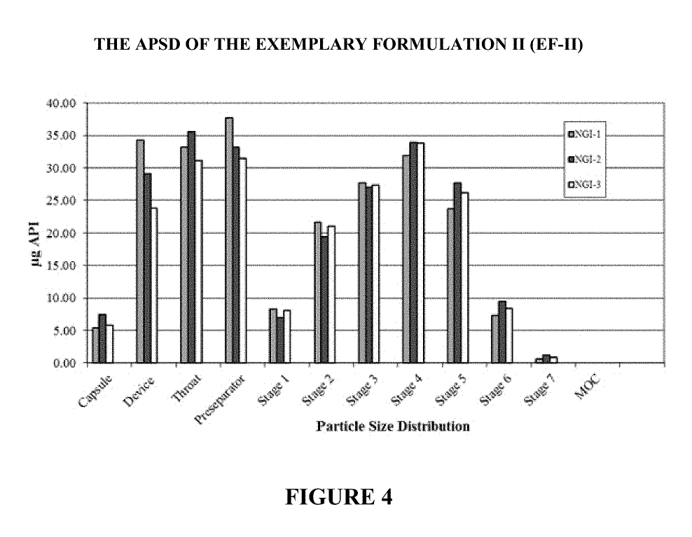

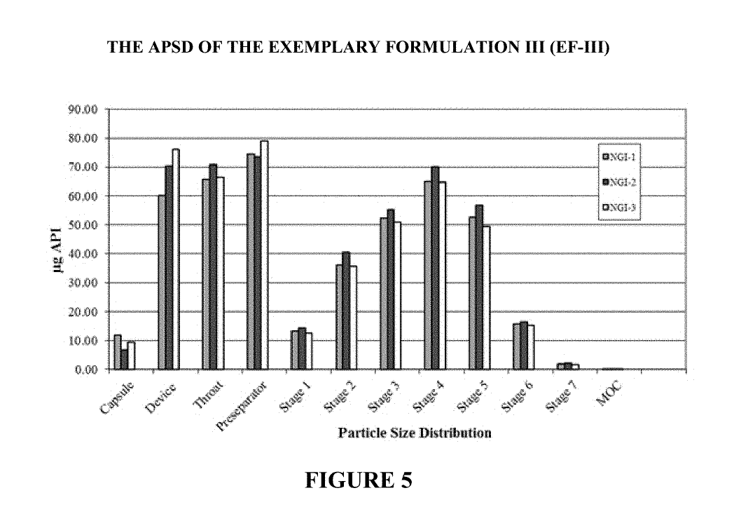

Aerodynamic particle size distribution (APSD) of individual NGI runs is given in FIGS. 3-5. Mean aerosol performance data are given in Table 2.

TABLE 2
The Mean APSD Parameters of EF-I, EF-II, EF-III Measured by NGI

| Blend | EF-I | EF-II | EF-III |
|---|---|---|---|
| Relative Humidity (%) | 46.0 | 46.0 | 46.0 |
| % Recovered | 89.9 | 94.0 | 98.4 |
| % RSD | 2.6 | 3.3 | 4.5 |
| Preseparator, % of Loaded | 19.3 | 14.1 | 16.1 |
| % RSD | 16.2 | 8.7 | 3.8 |
| Delivered Dose, % of Loaded | 74.2 | 79.4 | 81.8 |
| % RSD | 3.5 | 2.1 | 4.8 |
| Fine Particle Fraction (≤5 μm), % of Delivered | 47.8 | 52.7 | 52.1 |
| Dose | | | |
| % RSD | 7.4 | 3.9 | 1.9 |
| Mass Median Aerodynamic Diameter (μm) | 2.9 | 2.6 | 2.5 |
| % RSD | 5.3 | 4.9 | 0.3 |
| Geometric Standard Deviation | 2.2 | 2.1 | 2.1 |
| % RSD | 2.5 | 1.1 | 0.8 |

The FPF of EF-I, EF-II, and EF-III were 47.8%, 52.7%, and 52.1%, respectively. The MMAD of EF-I, EF-II, and EF-III under ambient conditions is 2.9 μm, 2.6 μm, and 2.5 μm, respectively.

### Example 7: Solubility of Palonosetron in pMDI Formulations

The solubility of palonosetron is measured in a pMDI medium of HFA 134a, HFA227, a mixture of HFA 134a and ethanol, as well as a mixture of HFA227 and ethanol.

From the foregoing description, various modifications and changes in the compositions and methods provided herein will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

1. An inhalation formulation comprising i) palonosetron or a pharmaceutically acceptable salt thereof, and ii) an excipient that comprises a fine powder and a coarse powder, and wherein the formulation exhibits a minimum fine particle fraction (FPF) of at least 40%; and wherein palonosetron is about 0.5% to about 4% of the total composition of formulation; and the fine powder and coarse powder are present in a mass ratio of about 1:14, 1:10, 1:9, 1:4, or 1:2.5.

2. (canceled)

3. The formulation of claim 1, wherein the fine powder and the coarse powder are lactose.

4. The formulation of claim 3, wherein the coarse powder has average particle size of about 50 to 60 or 65 to 85 μm.

5. The formulation of claim 3, wherein the fine powder has average particle size of about 1 to 5 μm.

6. (canceled)

7. (canceled)

8. (canceled)

9. (canceled)

10. (canceled)

11. (canceled)

12. (canceled)

13. (canceled)

14. The formulation of claim 1, wherein the palonosetron or a pharmaceutically acceptable salt thereof is present in an amount of about 0.6%, about 1%, about 2%, or about 4% of the total weight of the formulation.

15. The formulation of claim 1, wherein the pharmaceutically acceptable salt thereof is palonosetron hydrochloride.

16. (canceled)

17. The formulation of claim 1, wherein the formulation is suitable for pulmonary delivery.

18. (canceled)

19. (canceled)

20. (canceled)

21. The formulation of claim 1, wherein the formulation is in a form of powder.

22. (canceled)

23. (canceled)

24. (canceled)

25. (canceled)

26. (canceled)

27. (canceled)

28. (canceled)

29. (canceled)

30. (canceled)

31. (canceled)

32. The formulation of claim 1, wherein upon pulmonary delivery to a subject, the formulation exhibits an AUC of palonosetron equal or higher of that obtained following oral delivery of palonosetron.

33. (canceled)

34. (canceled)

35. (canceled)

36. A method of reducing or preventing nausea or vomiting in a subject, comprising administering the formulation of claim 1 to the subject.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method of making the formulation of claim 1, comprises first blending a fine powder of an excipient and a coarse powder of the excipient, a resulting mixture of which is further blended with palonosetron or a pharmaceutically acceptable salt thereof.

52. The formulation of claim 1, wherein the palonosetron or a pharmaceutically acceptable salt thereof is present in an amount of about 1.1%, or about 4% of the total weight of the formulation.

53. The formulation of claim 1, wherein the palonosetron or a pharmaceutically acceptable salt thereof is present in an amount of about 0.075 mg, about 0.25 mg, about 0.5 mg, or about 1 mg.

54. The formulation of claim 1, wherein the excipient is a mixture of fine and coarse powder lactose, and the fine powder and coarse powder are present in a mass ratio of about 1:9, 1:4, or 1:2.5.

55. The formulation of claim 1, wherein the formulation comprises about 0.5 mg of palonosetron or a pharmaceutically acceptable salt thereof, about 3.75 mg of fine lactose powder and about 8.75 mg of coarse lactose powder.

56. The formulation of claim 1, wherein the formulation comprises about 0.25 mg of palonosetron or a pharmaceutically acceptable salt thereof, about 2.5 mg of fine lactose powder and about 10 mg of coarse lactose powder.

57. The formulation of claim 1, wherein the formulation comprises about 0.075 mg of palonosetron or a pharmaceutically acceptable salt thereof, about 1.25 mg of fine lactose powder and about 11.25 mg of coarse lactose powder.

58. The formulation of claim 1, wherein the formulation has a fine particle fraction (FPF) of at least 55%.

59. The formulation of claim 1, wherein the formulation has a fine particle fraction (FPF) of at least 65%.

60. The formulation of claim 3, wherein the coarse powder has average particle size of about 60 to 90 μm.
